(19) United States
(12) Patent Application Publication
(10) Pub. No.: US 2018/0043070 A1
(43) Pub. Date: Feb. 15, 2018

(54) BREAST MILK COLLECTION SYSTEM WITH BAG ATTACHMENT

(71) Applicant: TARGET BRANDS, INC., Minneapolis, MN (US)

(72) Inventors: Michael Miller HABIG, Cincinnati, OH (US); Katherine LARSON, Minneapolis, MN (US); Samuel D. SILVA, Leonardtown, MD (US); Chad Robert KREYE, Woodbury, MN (US); Sara FRAILICH, Saint Paul, MN (US); Kathryn Lee SCOTT, Golden Valley, MN (US); Jennifer BERGERSON, Columbus, MN (US)

(73) Assignee: TARGET BRANDS, INC., Minneapolis, MN (US)

(21) Appl. No.: 15/674,210

(22) Filed: Aug. 10, 2017

Related U.S. Application Data
(60) Provisional application No. 62/373,472, filed on Aug. 11, 2016.

(51) Int. Cl. A61M 1/06; A61M 1/00; A61M 39/10; B65D 33/00; B65D 83/08; B65D 30/22; B65D 77/30; A61J 9/00; B65D 33/25

(52) U.S. Cl. CPC A61M 1/064; A61J 9/005; A61M 1/0019; A61M 39/1011; B65D 33/2508; B65D 83/0805; B65D 31/12; B65D 77/30; B65D 33/007; A61J 2200/76; A61J 2200/70; A61J 2205/30; A61M 2209/06; A61M 2039/1033

(57) ABSTRACT

A breast milk collection system includes a storage bag and at least one coupling member formed separately from the storage bag and defining a first side and a second side. The storage bag includes a storage chamber, a closure strip for selectively closing the storage chamber, two bag flaps each extending away from the closure strip, and two coupling components each coupled to a different one of the two bag flaps. Each of the two coupling components includes a plurality of flap protuberances. The first side of the at least one coupling member includes a plurality protuberances extending therefrom, and the second side of the at least one coupling member is at least partially covered with adhesive for selective application of the at least one coupling member to a breastpump apparatus. At least one of the two coupling components selectively couples with the at least one coupling member.

### CROSS-REFERENCE TO RELATED APPLICATION

This application is a non-provisional application of and claims priority under 35 U.S.C. §119 to U.S. Provisional Patent Application No. 62/373,472, filed Aug. 11, 2016, which is incorporated herein by reference.

### BACKGROUND OF THE INVENTION

The benefits of nourishing newborn infants by breastfeeding have long been established. Nursing mothers today are increasingly busy and for various reasons often wish to express breast milk to store for future use. A woman typically expresses breast milk using an electric or manual pumping device in connection with a funnel or shield that is held tightly over the woman's breast. The pumping device creates intermittent suction or pressure levels inducing milk flow and directing the flow of milk through the shield and to a storage bag coupled to the pumping device.

In typical breast milk collection systems, a breastpump apparatus expresses milk into a bottle or other similar hard-sided, reusable receptacle. However, breast milk is most commonly stored in a flexible storage bag. In these instances, the mother uses the breastpump to fill a bottle with breast milk, removes the bottle from the breastpump, empties the bottle into the bag for freezing, and then washes the bottle for future use. To decrease the time and hassle with this typical breast collection method, it is desirable to remove the reusable receptacle from the method entirely and to, instead, express milk directly into a storage bag such that milk transfer and receptacle cleaning steps are eliminated.

As nursing mother's increasingly collect breast milk for future use, their milk stockpile increases in size resulting in a need to store many bags of collected breast milk. In many instances, the storage, use, and organization of the bags of collected breast milk is problematic given the need for freezer space and a desire to have infants consume the oldest stored breast milk first. These tasks are complicated by the very busy lives of nursing mother's having little or no time to devote to organization of the stored breast milk.

### SUMMARY

A breast milk collection system includes a storage bag and at least one coupling member formed separately from the storage bag and defining a first side and a second side. The storage bag includes a storage chamber, a closure strip for selectively closing a top of the storage chamber, two bag flaps each extending from the closure strip away from the storage chamber, and two coupling components each coupled to a different one of the two bag flaps. Each of the two coupling components includes a plurality of hook protuberances extending from one of the two bag flaps toward the other one of the two bag flaps. The first side of the at least one coupling member includes a plurality protuberances extending therefrom, and the second side of the at least one coupling member is at least partially covered with adhesive for selective application of the at least one coupling member to a breastpump apparatus. At least one of the two coupling components selectively couples with the at least one coupling member via interaction between the plurality of hook protuberances of the at least one of the two coupling components and the plurality of protuberances of the at least one coupling member when of the at least one of the two coupling components and the at least one coupling member are pushed together. Other breast milk collection systems, bags, adapters, storage bags, assemblies, and associated methods are also described herein.

### BRIEF DESCRIPTION OF THE DRAWINGS

Embodiments of the invention will be described with respect to the figures, in which like reference numerals denote like elements, and in which:

FIG. 1 is a front perspective view illustration of a breast milk collection system, according to one embodiment of the present invention.

FIG. 2 is a front perspective view illustration of the breast milk collection system of FIG. 1 preparing for use, according to one embodiment of the present invention.

FIG. 3 is a front, perspective view illustration of the breast milk collection system of FIG. 1 during use, according to one embodiment of the present invention.

FIG. 4 is a top perspective view illustration of a portion of an inclined open bag from the breast milk collection system of FIG. 1 with detail, according to one embodiment of the present invention.

FIG. 5 is a top perspective view illustration of a storage bag, according to one embodiment of the present invention.

FIGS. 6A-6D each illustrate cross-sectional views of hook protuberances of differing shapes, according to different embodiments of the present invention.

FIG. 7 is a top perspective view an adhesive backed coupling member of the breast milk collection system of FIG. 1, according to one embodiment of the present invention.

FIG. 8 is a bottom perspective view the adhesive backed coupling member of FIG. 7, according to one embodiment of the present invention.

FIG. 9 is a cross-sectional view illustration and associated detail of the breast milk collection system and breastpump apparatus taken about line X-X in FIG. 3, according to one embodiment of the present invention.

FIG. 10 is a front perspective view of a breast milk collection bag, according to one embodiment of the invention.

FIG. 11 is a front perspective view illustration of a breast milk collection system including the breast milk collection bag of FIG. 10, according to one embodiment of the present invention.

FIG. 12 is a front perspective view illustration of a storage container, according to one embodiment of the present invention.

FIG. 13 is a front perspective view illustration of the storage container of FIG. 10 being used for breast milk storage, according to one embodiment of the present invention.

FIG. 14 is a front perspective view illustration of an adapter for use in a breast milk collection system, according to one embodiment of the present invention.

FIG. 15 is a front perspective view illustration of a breast milk collection system incorporating the adapter of FIG. 14, according to one embodiment of the present invention.

FIG. 16 is a front perspective view illustration of an adapter for use in a breast milk collection system, according to one embodiment of the present invention.

FIG. 17 is a front perspective view illustration of a breast milk collection system incorporating the adapter of FIG. 16, according to one embodiment of the present invention.

FIG. 18 is an exploded, front perspective view illustration of a breast milk collection system including an adapter, according to one embodiment of the present invention.

FIG. 19 is a front view of a bag for use with a breast milk collection system, according to one embodiment of the present invention.

FIG. 20 is a front view of the bag of FIG. 19 during storage, according to one embodiment of the present invention.

FIG. 21 is a front view of a bag for use with a breast milk collection system, according to one embodiment of the present invention.

FIG. 22 is a front perspective view of a bag for use with a breast milk collection system, according to one embodiment of the present invention.

FIG. 23 is a front perspective view of the bag of FIG. 22 during use as part of a breast milk collection system, according to one embodiment of the present invention.

FIG. 24 is a front perspective view of the bag of FIG. 22 during use after storage of breast milk therein, according to one embodiment of the present invention.

FIG. 25 is a front perspective view of the bag of FIG. 22 during use after storage of breast milk therein, according to one embodiment of the present invention.

FIG. 26 is a front perspective view of a bag for use with a breast milk collection system, according to one embodiment of the present invention.

### DETAILED DESCRIPTION

The following detailed description of the invention provides example embodiments and is not intended to limit the invention or the application and uses of the invention. Furthermore, there is no intention to be bound by any theory presented in the preceding background of the invention or the following detailed description of the invention. Relational terms herein such a first, second, top, bottom, etc. may be used herein solely to distinguish one entity or action from another without necessarily requiring or implying an actual such relationship or order. In addition, as used herein, the terms “about” or “substantially” apply to all numeric values or descriptive terms, respectively, and generally indicate a range of numbers or characteristics that one of skill in the art would consider equivalent to the recited values or terms, that is, having the same function or results.

This innovation provides breast milk collection system for use with a variety of breastpump apparatuses permitting milk to be expressed through via the breastpump and into a storage bag rather than a bottle or other typical re-usable container. In particular, in one embodiment of the innovation, the breast milk collection system includes a storage bag and at least one coupling member formed separately from the receptacle. The storage bag includes an integrated coupling component near an opening thereof that is configured to be selectively secured to the at least one coupling member upon the application of pressure thereto. In one embodiment, each of the coupling members includes an adhesive back or other readily couplable surface for ready attachment to a breastpump apparatus. In one example, the at least one coupling member and storage bag can be applied to any breastpump apparatus, whether manual, electric, a manual-electric hybrid, etc., effectively retrofitting the breastpump apparatus to facilitate expression of breast milk directly into a storage bag, rather than into a bottle or other container and then transferred into a storage bag. In one example, storage bag is selectively coupled to breastpump apparatus on at least two opposing and spaced apart locations of the storage bag to hold the storage bag in an open position for receiving breast milk. The storage bag includes an integrated seal to close the storage chamber in a liquid tight manner suitable for storage at room temperature, refrigerated temperatures, and below freezing temperatures.

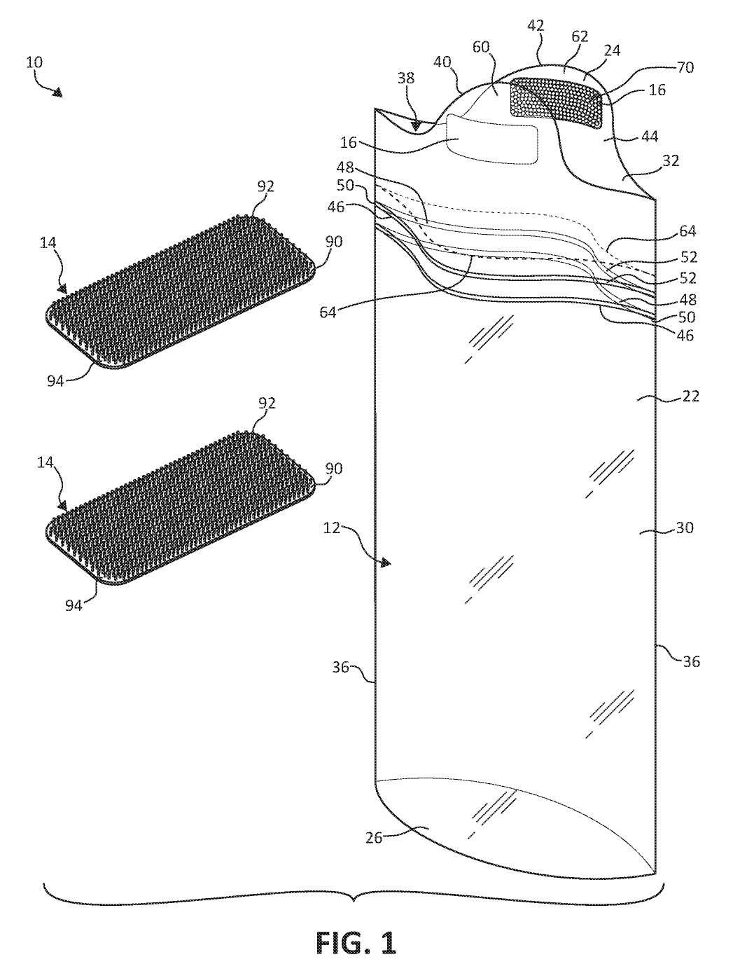

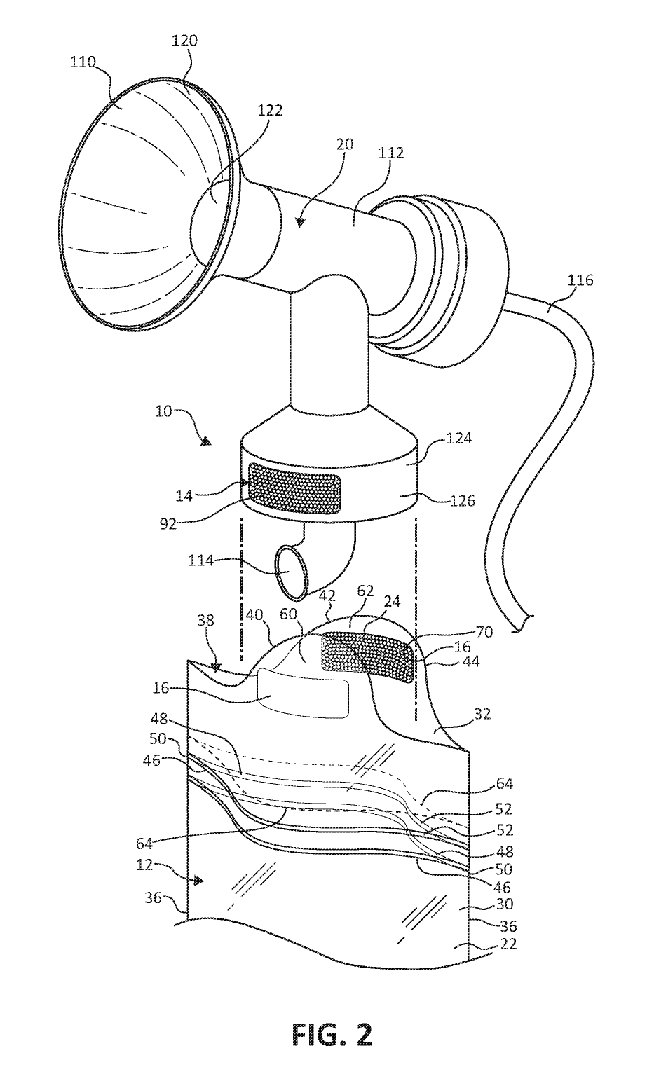

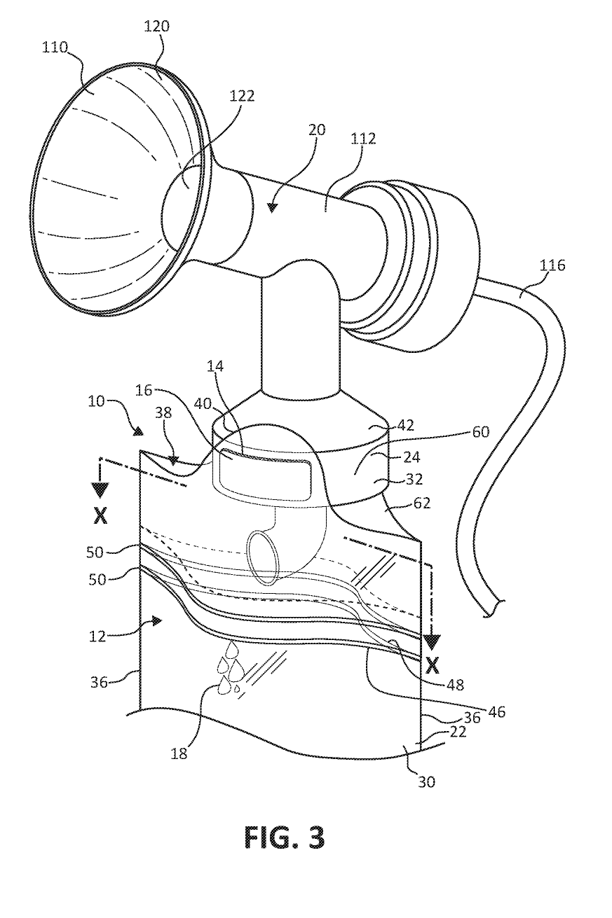

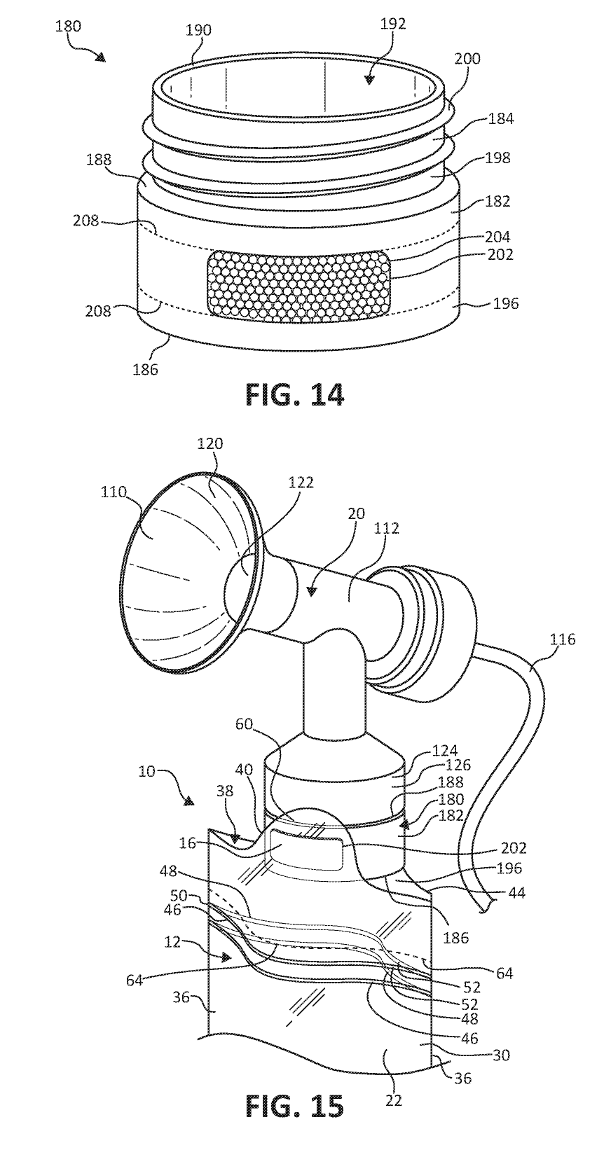

Turning to the Figures, FIG. 1 illustrates a breast milk collection system 10, according to one embodiment of the invention, including a storage bag 12, or other storage receptacle, and at least one coupling member 14 formed separately from the storage bag 12. The storage bag 12 includes at least one coupling component 16 readily and selectively securable to the at least one coupling member 14 in a manner capable of hanging storage bag 12 from the at least one coupling member 14 even when the when storage bag 12 is partially or completely filled with breast milk 18 (see, e.g., FIG. 3 and FIG. 14). Additionally referring to FIGS. 2 and 3, coupling member 14 is additionally configured for ready attachment to a breastpump apparatus 20 such that storage bag 12 is able to hang from breastpump apparatus 20 via coupling member 14. Breastpump apparatus 20 (see, e.g., FIGS. 2 and 3) is used to at least partially fill storage bag 12 while storage bag 12 hangs therefrom. Once filled to a desired level, storage bag 12 is pulled away from the coupling member 14, sealed, and stored for future use.

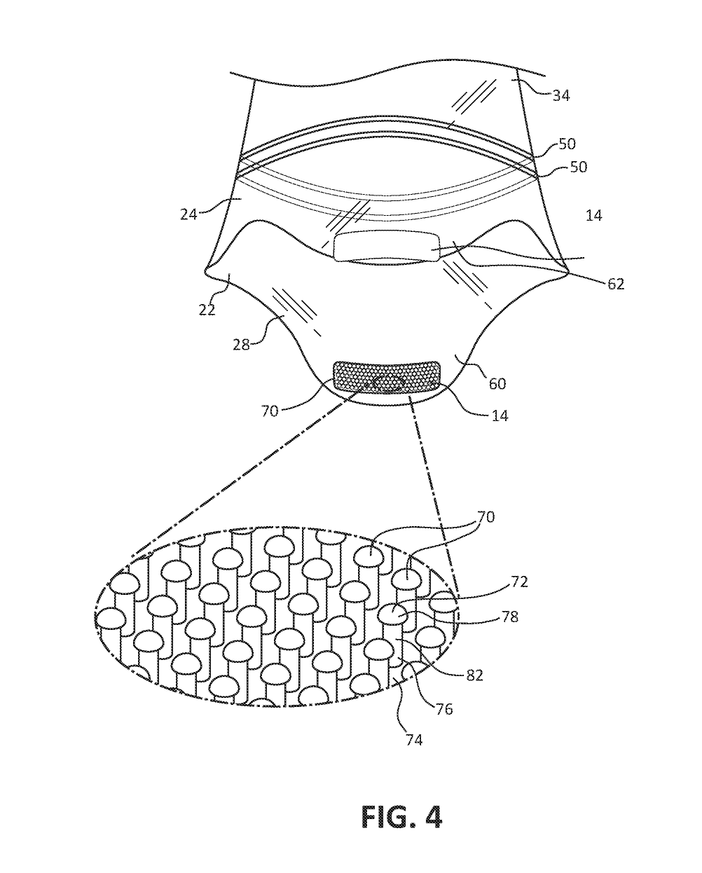

More specifically, in one embodiment, storage bag 12 is primarily formed of a flexible material configured for safe consumable product storage at range of temperatures for various storage options. In one example, storage bag 12 is substantially transparent while, in another embodiment, is entirely or at least partially opaque. Storage bag 12 includes a front wall 22, a rear wall 24, and a gusset or bottom wall 26, according to one embodiment of the present invention, and is configured to have a rectangular and substantially planar overall shape when folded into a closed position prior to use. Front wall 22 defines an interior surface 28 (see, e.g., FIG. 4) and an exterior surface 30 opposite interior surface 28. Similarly, rear wall 24 defines an interior surface 32 and an exterior surface 34 (see, e.g., FIG. 4) opposite interior surface 32. Interior surface 28 of front wall 22 is aligned with and placed atop interior surface 32 of rear wall 24 and opposing side seams 36 are formed along opposing sides thereof to secure front wall 22 with rear wall 24. In one example, side seams 36 are integrally formed where front wall 22 and rear wall 24 are formed of a single piece of substrate and no additional heat or other seal is applied thereto. In another example, side seams 36 are additionally or alternatively formed as heat, chemical, or other suitable watertight seals.

Bottom wall 26 is formed integrally with and/or coupled to, via a watertight seal, bottoms of each of front wall 22 and bottom wall 26 opposite top opening 44. Bottom wall 26, in one example, is formed as an oval or a vesica piscis, in other words, a football shape, allowing storage bag 12 to make use of initially planar, but flexible, front wall 22 and rear wall 24 and linear side seams 36, while still defining a storage chamber 38 therein. When storage bag 12 is folded for storage prior to filling, bottom wall 26 is folded up between front wall 22 and rear wall 24, as will be apparent to those of skill in the art. In one example, when storage bag 12 is folded and empty, each of front wall 22 and rear wall 24 lay substantially flat and planar with bottom wall 26 folded in between. When storage bag 12 is opened, while front wall 22 and rear wall 24 remain joined to each other along opposing side seams 36, front wall 22 and rear wall 24 bow outwardly relative to each other, due to the shape of bottom wall 26, forming storage chamber 38 therebetween adjacent to each of interior surfaces 28 and 32.

In one example, front wall 22, rear wall 24, and bottom wall 26 are each formed of a plastic material, such as polyethylene film or other suitable material, formed of single or multiple layers thereof. The plastic material is at least one of BPA-free, Consumer Product Safety Commission (CPSC) standard compliant, pre-sterilized, phthalate free, configured to reduce oxygen transmission therethrough, configured to be resistant to ultra-violet (UV) ray transmission, and polyvinyl chloride (PVC) free, in one embodiment. In one example, the plastic material forming storage bag 12 is BPA-free, CPSC standard compliant, pre-sterilized, phthalate free, configured to reduce oxygen transmission from storage bag 12, configured to be resistant to UV ray transmission, and PVC free. The plastic material is one of clear, translucent, and/or opaque.

Each of front wall 22 and rear wall 24 define a top edge 40 and 42, respectively, opposite bottom wall 26, such that top edges 40 and 42 collectively define a top opening 44 to storage bag 12. Each of front wall 22 and rear wall 24 also define at least one closure strip element 46 and 48, respectively, near to, but spaced downwardly, that is, positioned away from the corresponding one of top edges 40 and 42, respectively, toward bottom wall 26. In one embodiment, closure strip elements 46 and 48 are each of a different one of an elongated male and an elongated female or other corresponding type closure strip elements 46 and 48 formed in interior surfaces 28 and 32 to face the other of interior surfaces 28 and 32. Closure strip elements 46 and 48 are configured to selectively mate with each other forming a closure strip 50, which selectively couples and uncouples top edges 40 and 42 of storage bag 12 with one another.

In one example, front wall 22 includes two closure strip elements 46 in linear form, one above, that is, nearer top opening 44, than the other, and rear wall 24 includes two corresponding closure strip elements 48 in linear form, one above, that is, nearer top opening 44 to collectively form a double seal or double closure strip 50 for an even more secure closure of top opening 44 than a single closure strip 50. Each closure strip element 46 is positioned a distance away from bottom wall 26 substantially equal to a corresponding closure strip element 48, such that even when closure strip elements 46 are coupled to corresponding closure strip elements 48, storage bag 12 lays substantially flat. Closure strip 50 is any suitable closure means having a selectively water tight seal and, in one example, is a zip-top, slider bar, hook-and hook, hook and loop or other suitable press-and-seal coupling strip, as will be apparent to those of skill in the art.

In one example, each closure strip element 46 and 48 includes a pair, plurality, or series of corresponding projections or depressions, generally indicated in FIG. 1 at 52, disposed adjacent to one another configured to engage with the corresponding projections or depressions 52 of the other one of closure strip element 46 and 48 when closure strip elements 46 and 48 are pressed together to securely close top opening 44 from one of opposing side seams 36 to the other one of opposing side seams 36. Projections or depressions 52 of one closure strip element 46 have shapes that facilitate the engagement of closure strip 50 when closure strip elements 46 and 48 are pressed toward each other and disengagement of closure strip 50 when closure strip elements 46 and 48 are peeled away from each other, as will be apparent to one of skill in the art upon reading this application. In one example, each of the projections or depressions 52 are linear in overall shape forming a single or plurality of zip-top closure lines, e.g., two zip-top closure lines. The one or more closure strips 50 securely hold any breast milk or other suitable liquid in storage bag 12 for storage, e.g., at room temperature, at higher ambient temperature, at refrigerated temperatures, and/or at freezing temperatures.

In one embodiment, each of front wall 22 and rear wall 24 of storage bag 12 defines a flap 60 or 62, respectively, each extending from closure strip 50 to a respective one of top edge 40 and 42, respectively. Each flap 60 and 62 may extends freely from closure strip and/or each flap 60 and 62 may include at least a portion joined to the other one of flaps 60 and 62 along side seams 36. In one example, each flap 60 and 62 is configured to facilitate expression of breast milk into storage chamber 38 and/or removal of breast milk from storage chamber 38, as will be further described below. In one embodiment, flaps 60 and 62 also are used to maintain information about any breast milk contained therein, as will be further described below. Flaps 60 and 62, in one example, each define a perforation line 64 just above, that is nearer a corresponding top edge 40 or 42 of storage bag 12. Perforation lines 64 facilitate ready removal of flaps 60 and 62, if desired, such that flaps 60 and 62, and any coupling components 16 located on flaps 60 and/or 62 do not impede pouring of milk out of storage bag 12.

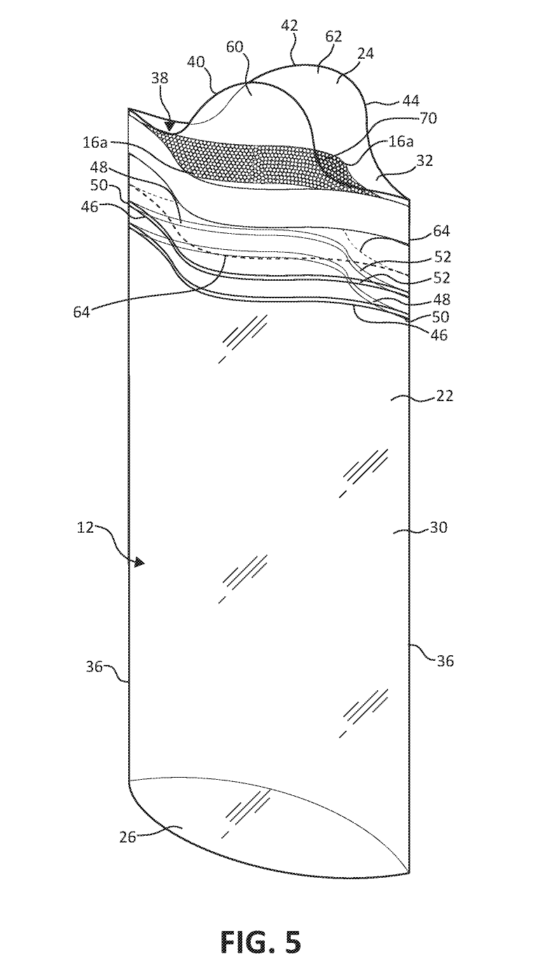

Each coupling component 16 of storage bag 12 is secured to and/or integrally formed with one of front wall 22 and rear wall 24, for example to flaps 60 and 62 of front wall 22 and/or rear wall 24. As primarily illustrated in the figures, in one embodiment, each coupling component 16 extends across less than half, and in one example, less than a quarter, of an outside width of storage bag 12 measured between side seams 36. However, in one embodiment, coupling component 16A extends across a substantial entirety of the outside width of storage bag 12 as illustrated, for example, in FIG. 5. It should be understood that unless specifically described otherwise herein, descriptions of coupling component 16 hold true for other coupling component, such as coupling component 16A, other than the dimension of each coupling component 16 or 16A relative to the outside width of storage bag 12.

In one example, coupling component 16 is configured to selectively couple storage bag 12 with breastpump apparatus 20 (see, e.g., FIG. 2) via engagement with the at least one coupling member 14. Each coupling component 16 is formed on a corresponding flap 60 of front wall 22 or rear wall 24 between and spaced from each of the one or more coupling strips 50 and top edge 40 or 42 of the corresponding one of front wall 22 and rear wall 24. While each coupling component 16 may have a width extending from one of opposing side seams 36 to the other, in one example, each coupling component 16 has a width that is smaller than a width of storage bag 12 and is substantially centered between and spaced from each of opposing side seams 36, as illustrated, for example, in FIG. 1.

Additionally referring to FIG. 4, in one embodiment, each of coupling components 16 is a suitable press-and-seal closure portion formed along interior surface 28 of front wall 22 or interior surface 32 of rear wall 24 to face the other of interior surface 28 and interior surface 32. More specifically, in one example, coupling components 16 are each a part of a two-part hook type, pressable fastener, such as a hook-and-loop or hook-and-hook fastener. In one embodiment, coupling components 16 are hook-and-hook fastening components formed, at least in part, by being integrally molded into, blown film formed, extruded, heat sealed to, and/or otherwise suitably formed integrally with or coupled to front wall 22 and rear wall 24. In one example, one of coupling components 16 and one of coupling members 14 collectively form a fastener, such as a Press-Lok® 731 fastener, as offered by Velcro Companies, having offices in Manchester, N.H., an equivalent, or variation thereof; however, use of other suitable fasteners is also contemplated. Two-part hook type, pressable fasteners provide for easy fastening between two coupling components 16 or one of coupling component 16 and one of coupling members 14 (e.g., FIGS. 1-3) without the use of tools resulting in a coupling that is both secure and easily unfastened when desired by a consumer.

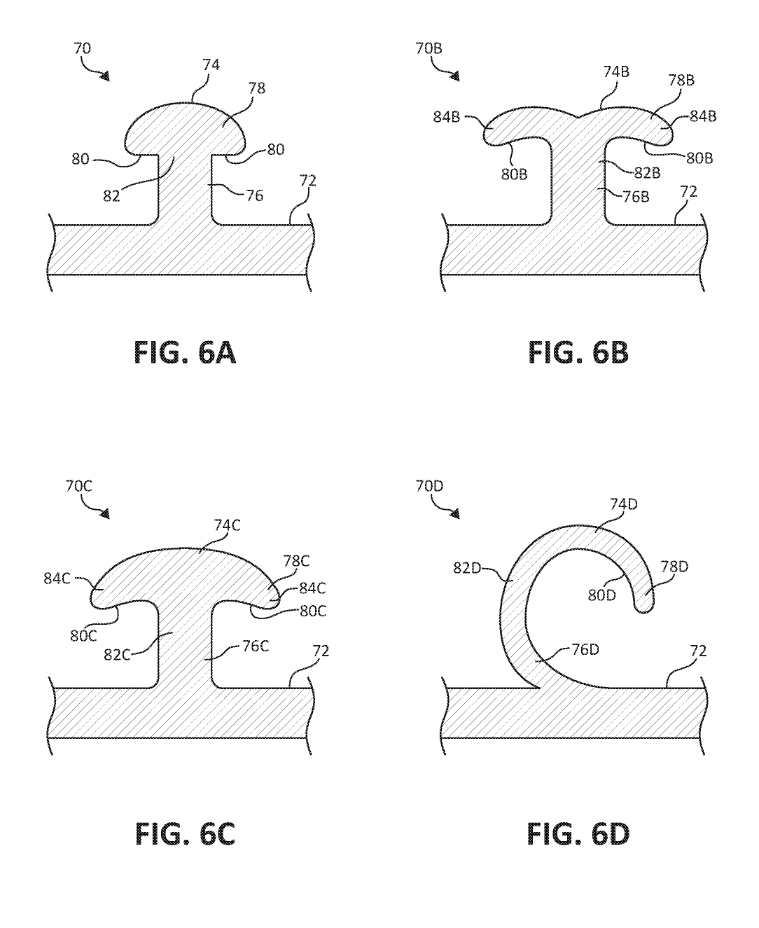

The detail portion of FIG. 4 and FIG. 6A more closely illustrate the structure of one example of a hook type fastener, more specifically of a hook-and-hook type fastener, including a plurality of hook protuberances 70 extending from a base surface 72, for example, where base surface 72 is one of interior surface 28 of front wall 22 or interior surface 32 of rear wall 24, to a free end 74 opposite base surface 72. The plurality of hook protuberances 70 generally extend from base surface 72 in an array of columns and rows, while, in other embodiments, the plurality of hook protuberances 70 is less regular and/or linear. In one example, each of the plurality of hook protuberances 70 is integrally formed with front wall 22 and/or rear wall 24 of storage bag 12. In one embodiment, the plurality of hook protuberances 70 are formed of polypropylene and/or polyethylene and are molded into storage bag 12.

Each of the plurality of hook protuberances 70 is substantially identical, in one example, defining a stem or shaft 76 topped with a bulbous head 78, having a greater overall diameter than shaft 76. In this manner, undersides of bulbous head 78, surrounding an end of shaft 76 opposite base surface 72, define rails 80 for interacting with similar rails 80 on other corresponding coupling components 16 or coupling members 14, as will be further described below. Each shaft 76 is elongated and flexible in a manner allowing the plurality of hook protuberances 70 to flex under pressure to couple with or uncouple from coupling member 14. In other embodiments, the plurality of hook protuberances 70 includes hook protuberances 70 of various shapes and/or profiles.

While FIG. 4 illustrates one profile or shape of the plurality of hook protuberances 70, many other suitable shapes are contemplated. For instance, FIG. 6B illustrates a cross-sectional view of a hook protuberance 70B defining a stem or shaft 76B topped with a hooked head 78B, having a greater overall width than a diameter of shaft 76B. In this manner, undersides of hooked head 78B, surrounding an end 82B of shaft 76B opposite base surface 72, define rails 80B for interacting with similar rails on other corresponding coupling component or members. More specifically, in one example, hooked head 78B includes two tubular hooks 84B each extending away and downwardly from end 82B of shaft 76B in a different radial direction.

FIG. 6C illustrates a cross-sectional view of a hook protuberance 70C defining a stem or shaft 76C topped with a hooked head 78C, having a greater overall width than a diameter of shaft 76C. In this manner, undersides of hooked head 78C, surrounding an end 82C of shaft 76C opposite base surface 72, define rails 80C for interacting with similar rails on other corresponding coupling component or members. More specifically, in one example, hooked head 78C includes two, somewhat flattened tubular hooks 84C each extending away and downwardly from end 82C of shaft 76C in a different radial direction to collectively define a rounded top surface thereof.

FIG. 6D illustrates a cross-sectional view of a hook protuberance 70D defining a stem or shaft 76D topped with a hooked head 78D, having a greater overall width than a diameter of shaft 76D. In one example, shaft 76D extends from base surface 72 with a slight curvature. Hooked head 78D define a hook as a continued curvature of shaft 76d such that shaft 76D and hooked head 78D collectively define a C-shaped cross section, in one example. An underside of hooked head 78D extending from an end 82C of shaft 76C opposite base surface 72 defines a rail 80D for interacting with similar rails on other corresponding coupling component or members.

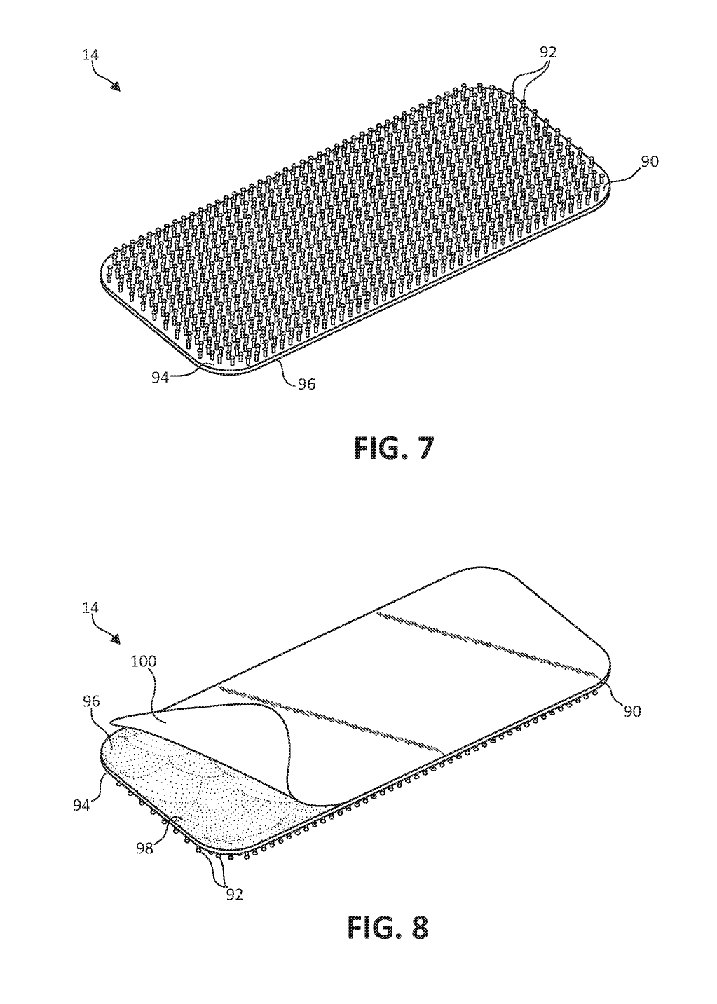

One example of coupling member 14 is shown in additional detail in the top and bottom perspective views of FIGS. 7 and 8. As illustrated, coupling member 14 includes a base substrate 90 and a plurality of hook protuberances 92 extending away from base substrate 90. Base substrate 90 is formed as a substantially planar, yet flexible piece of material defining a first primary side 94 and a second primary side 96 facing in a direction opposite that of first primary side 94. Base substrate 90 is suitably sized to extend along a limited portion of breastpump apparatus 20 as illustrated where two or more coupling members 14 are used. In one example, where only one coupling member 14 is used, it is sized with a length sufficient to extend around at least about 180 degrees of an outer perimeter of a lower portion of breastpump apparatus 20, as will be further described below.

Hook protuberances 92 each are coupled to base substrate 90 and extend away from base substrate 90 in suitable shape, for example, with any of the shapes described above with respect to hook protuberances 70, 70B, 70C, and 70D. In one embodiment, the plurality of hook protuberances 92 are each sized and/or shaped substantially identical to the plurality of hook protuberances 70, 70B, 70C, and 70D that are included in coupling component 16.

In one example, the plurality of hook protuberances 92 are arranged relative to base substrate 90 as an array in a series of columns and rows, while, in another example, the plurality of hook protuberances 92 are more randomly positioned and/or are positioned in locations offset from one another. In one example, the plurality of hook protuberances 92 are arranged and spaced from one another in an arrangement substantially identical to or otherwise complimentary to the arrangement of the plurality of hook protuberances 70 on storage bag 12, as will be further described below.

In one example, second primary surface 96 is at least partially covered with an adhesive 98 and a readily removable liner 100 is selectively placed over adhesive 98 until coupling member 14 is ready for use. Adhesive 98 is any suitable fastening agent configured to securely hold coupling member 14 to breastpump apparatus 20 (see, e.g., FIGS. 2 and 3) and, in one example, is a pressure sensitive adhesive of sufficient strength to prevent or at least greatly decrease inadvertent removal or peel back of an adhered coupling member 14 from the corresponding breastpump apparatus 20.

Referring once again to FIG. 2, breastpump apparatus 20 includes a breast shield 110, a housing 112, an outlet valve 114, and a pump connection line 116 coupled with a pump (not shown) of breastpump apparatus 20 configured to introduce intermittent suction to the apparatus and a woman's breast causing milk to be expressed therefrom. Breast shield 110 includes a funnel-shaped cup 120, in one embodiment, and is configured to fit adjacent with and engage at least a portion of a woman's breast (not shown). Breast shield 110 further includes an open channel 122 extending from an interior of funnel-shaped cup 120 for directing milk expressed from the breast to housing 112. Breast shield 110 is selectively coupled to housing 112 such that channel 122 is in open communication with a conduit system (not shown) formed within housing 112 and configured to direct milk expressed from the breast, through the conduit system, and to outlet valve 114. Outlet valve 114 is configured to open and close to allow suction to be created within housing 112 and breast shield 110 and to allow breast milk 18 (see FIG. 3) to be released for collection and storage.

In one example, housing 112 defines a coupling sleeve 124 just above and extending around outlet valve 114. Coupling sleeve 124 is generally internally threaded and/or otherwise formed to receive a top of a bottle or other container (not shown) having a correspondingly threaded neck (not shown), as will be apparent to those of skill in the art. In other embodiments, coupling sleeve 124 is not internally threaded. Coupling sleeve 124 generally has an outer surface 126, which is not used in coupling typical bottles or other containers to breastpump apparatus 20, and typically has a cylindrical or other suitably shape.

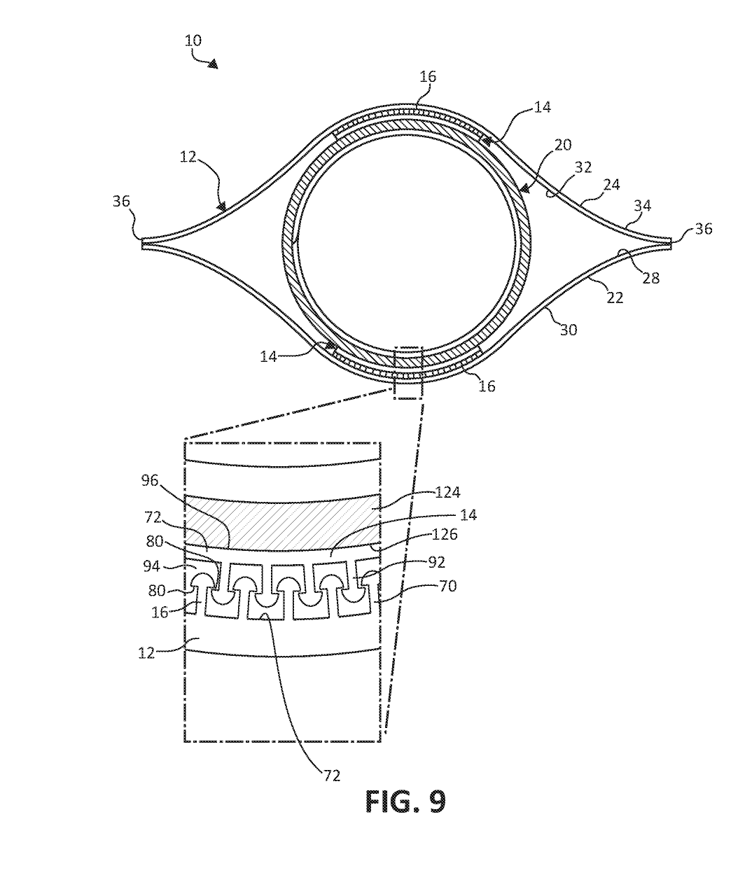

During use of breast milk collection system 10 each of the at least one coupling member 14 is secured to outer cylindrical surface 126 of coupling sleeve 124, for example, as illustrated with continued reference to FIG. 2. For example, coupling member 14 is sized to have a height less than an outer cylindrical surface 126 of a large number of brands and models of breastpump apparatus 20. During use, liner 100 of coupling member 14 is removed, and each coupling member 14 is adhered to outer cylindrical surface 126 such that a length of coupling member 14 extends along a portion of an outer perimeter of outer cylindrical surface 126. In one example, more than one coupling member 14 is applied to outer cylindrical surface 126, such as two coupling members 14, for instance, being coupled to outer cylindrical surface 126 on opposing sides thereof as shown in the cross-sectional view of FIG. 9.

Once coupling members 14 are applied to breastpump apparatus 20, breastpump apparatus 20 is configured for use with one of storage bags 12. For use, storage bag 12 is opened, separating flap 60 and flap 62 from one another. Top opening 44 of storage bag 12 is generally aligned with coupling sleeve 124. Storage bag 12 and breastpump apparatus 20 are moved relative to each other placing coupling component 16 formed by front wall 22 into vertical and horizontal alignment with one of coupling members 14, which is secured to coupling sleeve 124 of breastpump apparatus 20. Pressing on flap 60 over coupling component 16 and toward coupling sleeve 124 of breastpump apparatus 20 flexes the plurality of hook protuberances 70 and/or 92 until some of rails 80 formed by the plurality of hook protuberances 70 are pushed past heads 78 of the plurality of hook protuberances 92 and vice versa such that rails 80 of the plurality of hook protuberance 70 are placed to be adjacent rails 80 of hook protuberances 92, as illustrated in the detail portion of FIG. 9.

Similarly coupling component 16 formed by rear wall 24 of storage bag is placed into vertical and horizontal alignment with an opposite one of coupling members 14, which is also secured to coupling sleeve 124 of breastpump apparatus 20. Pressure is applied to coupling component 16 toward coupling sleeve 124 flexing the plurality of hook protuberances 70 and/or 92 of the corresponding coupling member 14 and coupling component 16 until some of rails 80 formed by the plurality of hook protuberances 70 are pushed past heads 78 of the plurality of hook protuberances 92 and vice versa such that rails 80 of the plurality of hook protuberance 70 are placed to be adjacent rails 80 of hook protuberances 92, as illustrated in the detail portion of FIG. 9.

The interaction between rails 80 of the plurality of hook protuberances 70 and the plurality of hook protuberances 92 repeated for the relatively larger numbers of hook protuberances 70 and 92 on each of the two corresponding coupling components 16 and one or more coupling members 14 effectively maintains each coupling member 14 secured to the corresponding coupling component 16 of storage bag 12 with at least a sufficient bond to collectively maintain a storage bag 12 suspended from coupling sleeve 124 of breastpump apparatus 20 even when storage bag 12 is filled with breast milk 18 (FIG. 2). Conversely, the flexible shafts 76 of hook protuberances 70 and 92 allow for interaction between rails 80 of coupling components 16 and coupling members 14 to be overcome when coupling component 16 of storage bag 12 is pulled away from the corresponding coupling member 14 allowing storage bag 12 to be separated from breastpump apparatus 20.

When storage bag 12 is so coupled with breastpump apparatus 20, in one embodiment generally illustrated, for example, in FIG. 3, breastpump apparatus 20 remains largely above or just below closure strips 50. By maintaining breastpump apparatus 20 in a relatively high position in storage bag 12 rather than largely within storage chamber 38 below closure strips 50, it decreases the likelihood that a consumer will overfill the bags in a manner that could result in breast milk 18 entering the tubing and other functional components of breastpump apparatus 20, which can be detrimental to the functionality and lifespan of breastpump apparatus 20. While storage bag 12 hangs from breastpump apparatus 20, a consumer places breast shield 110 over her breast and activates the breastpump apparatus 20, causing breast milk 18 to be expressed, via breastpump apparatus 20, directly into storage bag 12. In one embodiment, a consumer may express from more than one breast at the same time using a second breast shield 110, housing 112, storage bag 18 assembly. The consumer continues to express breast milk 18 into storage bag 12 until storage bag 12 is filled to a desired level taking care not to fill storage bag 12 over capacity and/or to otherwise allow breast milk 18 in storage bag 12 to infiltrate the tubing and other internal components

Once storage bag 12 is filled to a desired or end level, the consumer peels the flaps 60 and 62 away from coupling sleeve 124 separating protuberances 70 of coupling component 16 from protuberances 92 of coupling member 14. Once coupling components 16 are separated from coupling components 14, storage bag 12 is moved away from breastpump apparatus 20. The filled storage bag 12 is sealed using coupling strips 50, safely securing breast milk 18 therein for storage and/or future use. Clean up is limited to cleaning components of breastpump apparatus 20 as there is no intermediary storage container other than the end bag or other storage bag 12, which can be placed in the refrigerator for short term and/or freezer for long term storage of the breast milk 18 contained therein. As such, some of the time-consuming chores or hassle associated with expressing and storing breast milk 18 is eliminated. In one embodiment, flaps 60 are substantially removed from storage bag 12 by tearing storage bag 12 along perforation lines 64 prior to pouring breast milk 18 out of storage bag 12. In this manner, breast milk 18 is more easily poured into a bottle or other container without impedance from coupling components 16 on flaps 60 and 62.

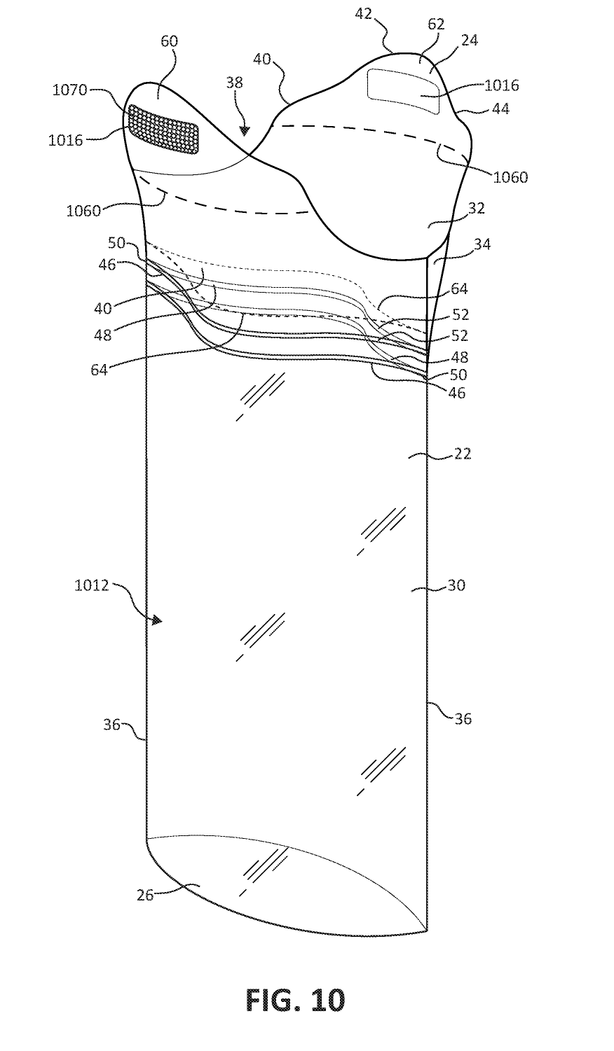

FIG. 10 illustrates one embodiment of a storage bag 1012 being substantially identical to any embodiment of storage bag 12 described above, other than the specific differentiated features recited herein. Storage bag 1012 includes coupling components 1016, which are substantially identical to coupling components 16. For example, coupling components 1016 are each a part of a two-part hook type, pressable fastener, such as a hook-and-loop or hook-and-hook fastener. In one embodiment, coupling components 1016 are hook-and-hook fastening components formed, at least in part, by being integrally molded into, blown film formed, extruded, heat sealed to, and/or otherwise suitably formed integrally with or coupled to front wall 22 and rear wall 24 of storage bag 1012. In one example, one of coupling components 1016 and one of coupling members 14 collectively form a fastener, such as a Press-Lok® 731 fastener, as offered by Velcro Companies, having offices in Manchester, N.H., an equivalent, or variation thereof; however, use of other suitable fasteners is also contemplated. Two-part hook type, pressable fasteners provide for easy fastening between two coupling components 1016 or one of coupling component 1016 and one of coupling members 14 (e.g., FIGS. 1-3) without the use of tools resulting in a coupling that is both secure and easily unfastened when desired by a consumer.

Unlike storage bag 12, in one embodiment, storage bag 1012 includes coupling components 1016 in a different placement than coupling components 16. More specifically, coupling components 1016 are coupled to, e.g., attached to or formed integrally with, exterior surface 30 of front wall 22 and exterior surface 34 of rear wall 24, such that coupling components 1016 initially face away from each other. In one example, placement of coupling components 1016 on exterior surfaces 30 and 34 of storage bag 1012 eases manufacturing of storage bag 1012. In one embodiment, storage bag 1012 defines a fold line 1060, which may be pre-defined and/or defined upon folding storage bag 1012 for use, that extends substantially parallel to closure strip elements 46 and 48 across each of front wall 22 and rear wall 24 between coupling component 1016 and top ones of closure strip elements 46 and 48.

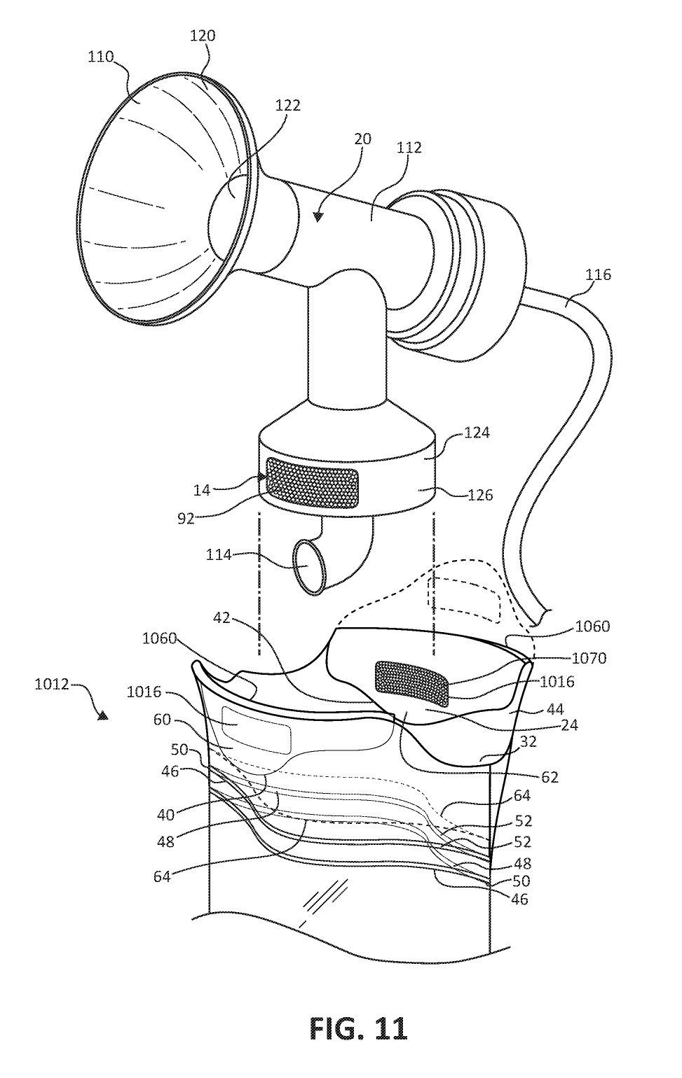

Referring to FIG. 11, during use of storage bag 1012, each of front wall 22 and rear wall 24 is folded about its respective fold line 1060 transitioning closure components 1016 from facing away from each other to face toward one another. Once positioned to face inwardly, each closure component 1016 is coupled to a corresponding closure member 14 of breastpump apparatus 20 in a similar manner as described with respect to closure components 16 above. Once storage bag 1012 is coupled to breastpump apparatus 20, breastpump apparatus 20 is used to express milk from a woman's breast directly into storage bag 1012 in much the same way as described above for storage bag 12.

Once storage bag 1012 is filled to a desired or otherwise resulting level, the consumer peels the flaps 60 and 62 away from coupling sleeve 124 separating protuberances 70 of coupling component 1016 from protuberances 92 of corresponding ones of coupling members 14. Once coupling components 1016 are separated from coupling components 14, storage bag 12 is moved away from breastpump apparatus 20 and front wall 22 and rear wall 24 are unfolded. Like filled storage bag 12, the filled storage bag 1012 is sealed using coupling strips 50, safely securing breast milk 18 therein for storage and/or future use. Clean up is limited to cleaning components of breastpump apparatus 20 as there is no intermediary storage container other than the end bag or other storage bag 1012, which can be placed in the refrigerator for short term and/or freezer for long term storage of the breast milk 18 contained therein. As such, some of the time-consuming chores or hassle associated with expressing and storing breast milk 18 is eliminated. In one embodiment, flaps 60 are substantially removed from storage bag 1012 by tearing storage bag 1012 along perforation lines 64 prior to pouring breast milk 18 out of storage bag 1012. In this manner, breast milk 18 is more easily poured into a bottle or other container without impedance from coupling components 1016 on flaps 60 and 62.

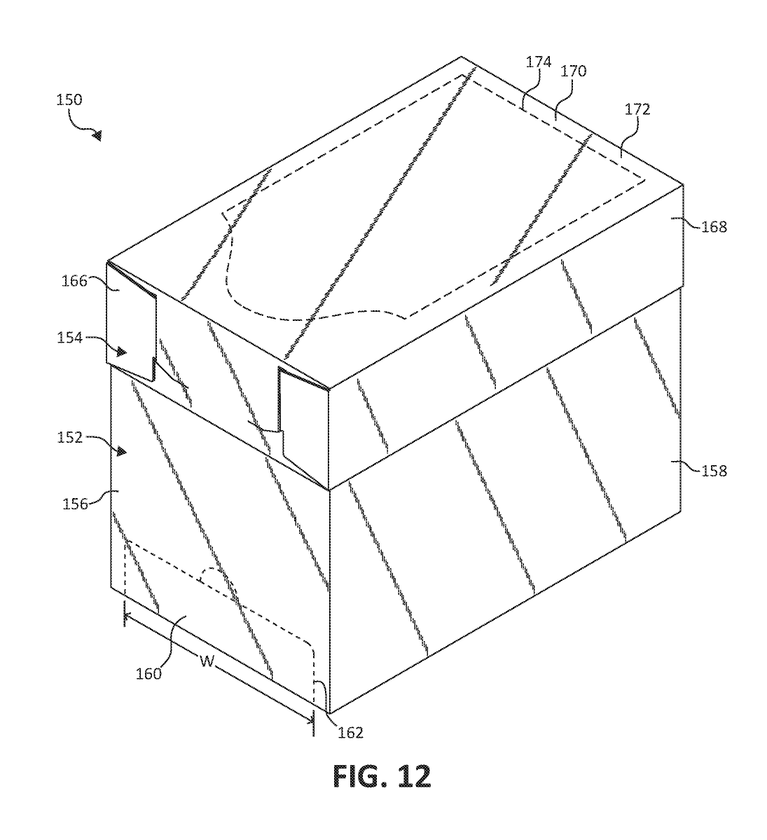

To further facilitate breast milk storage, in one embodiment, storage bags 12 are sold in a box 150 or other container that also is configured to hold filled storage bags 12 in a freezer, as illustrated with reference to FIGS. 11 and 12. Box 150 includes a base portion or body 152 and a cover 154, which, in one embodiment, are separately formed. Body 152 defines a front wall 156 and a series of sidewalls 158 forming a closed structure with a bottom wall (not shown) and a storage compartment 164 therein. In one embodiment, front wall 156 and sidewalls 158 are each substantially planar and define box 150 to have a width and length slightly larger than a width and length of storage bags 12, which will be stacked and stored therein. In one example, body 152 is formed of a single piece of planar material folded into a use position defining storage compartment 164.

In one example, front wall 156, includes a door 160 formed as a single piece with a remainder of front wall 156, defined by perforation lines 162. Door 160 is formed in front wall 156 immediately adjacent the bottom wall of box 150 such that perforation lines 162 forms three sides of door 160 while the bottom wall of box 150 defines the forth side of door 160. Door 160 has a width W, measured from side to side, that is greater than a width of frozen storage bag 12 such that storage bag 12 can be readily pulled from storage compartment 164 via door 160 as shown, for example, in FIG. 12. Perforation lines 162 are broken and door 160 is pushed into box 150 to define an opening to storage compartment 164, for example, as is also shown in FIG. 12.

Box body 152 is formed of any suitable material such as paperboard, corrugated material (e.g., micro flute material), other paper-based product, plastic, etc. In one example, box body 152 is formed of a polycarbonate-coated Scround. Box body 152 may be sold to consumers is an erected configuration, e.g., with empty storage bags 12 therein, and/or in a flattened configuration that is readily erected without the need for tools and/or fastening members, as will be apparent to those of skill in the art upon reading this application.

Cover 154 is sized and shaped to cover storage compartment 164 opposite the bottom wall (not shown). In one example, cover 154 includes a front wall 166 and additional sidewalls 168 with a top wall 170 extending from and between each of front wall 166 and additional sidewalls 168. While cover 154 is illustrated as being formed separately from body 152, in one example, cover 154 may be formed as a single piece with body 152, e.g., coupled with body 152 via a living hinge or fold line. In one example, cover 154 is formed of a single piece of planar material folded into a use position for covering body 152. Front wall 166 and other sidewalls 168 collectively surround an outer perimeter of body 152.

In one example, top wall 170 of cover 154 defines a top surface 172 that is substantially planar. The planarity of top surface 172 provides a surface for maintaining storage bag 12 filled with liquid-form breast milk 18 for freezing, since breast milk 18 maintained in a substantially flat manner in storage bag 12 during freezing will result in a flatter frozen shape that is easier to store, transport, etc. than a more bulky shape. In one example, top surface 172 of top wall 170 includes a graphical indication 174, for instance, an outline of a storage bag shape and or other indications to inform a consumer that top surface 172 is configured to support storage bag 12 while breast milk 18 therein is frozen.

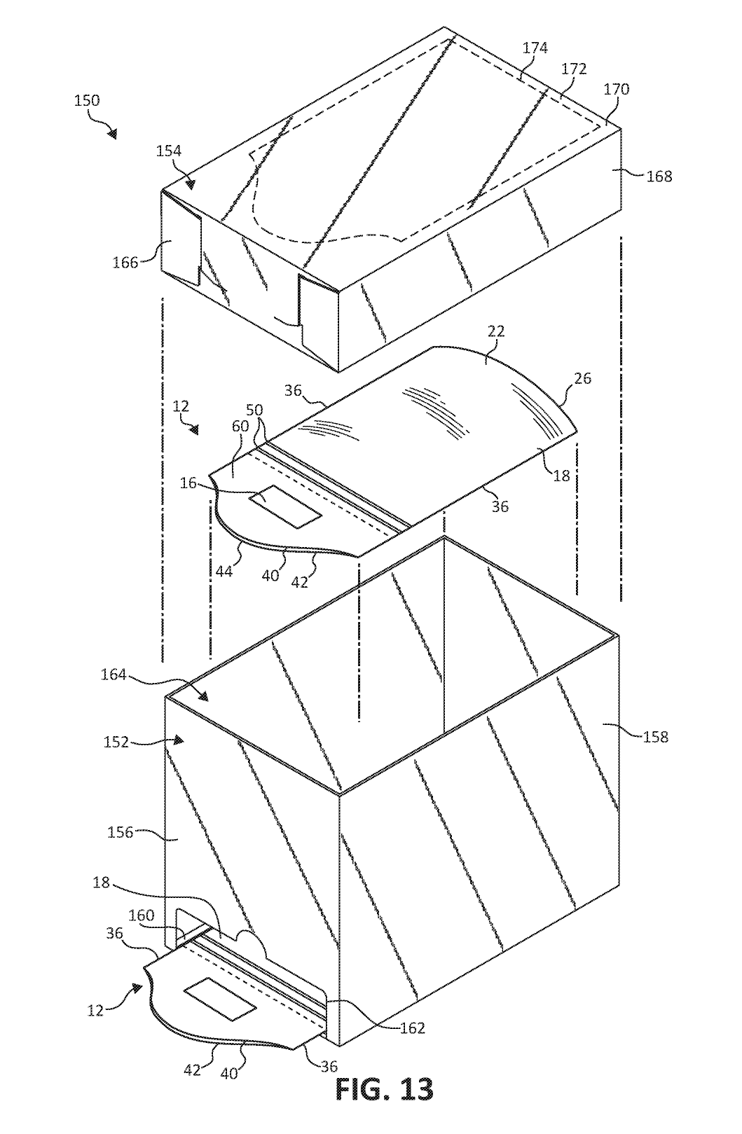

Box 150 is not only is used to hold a stack of empty storage bags 12 for sale, but also to hold filled storage bags 12 in a first-in-first-out arrangement. More specifically, referring primarily to FIG. 13, once storage bags 12 filled with breast milk 18 are frozen on top surface 172 or elsewhere, cover 154 is opened to allow the frozen storage bag 12 to be placed in storage compartment 164 on top of the bottom wall and any other filled storage bag 12 previously placed in box 150. When a consumer wishes to use the contents of a storage bag 12, within box 150, the bottommost storage bag 12 is withdrawn from box 150 via door 160, not via the top thereof. In this manner, the oldest filled storage bag 12 is removed from box 150 first, which is generally desirable to best maintain the freshness and nutrient value of the stored breast milk 18. While box 150 can be sold with storage bags 12, such as the packaging containing storage bags 12, in one example, box alternatively and/or additionally is sold in a similar or larger size for use in storing separately purchased storage bags 12. In one embodiment, box 150 is formed of a blank and is folded flat for in-store display configured to be easily erected by the end consumer without the use of tools.

Other solutions for converting typical breastpump apparatus to a direct pump to storage bag system are also contemplated. For example, referring to FIGS. 14 and 15, in one example, rather than securing, e.g., adhering, the at least one coupling member 14 directly to breastpump apparatus 20, as described above, an adapter 180 is used. Adapter 180 includes a cuff 182 and a neck 184 coaxially positioned and extending upwardly from cuff 182. More specifically, in one example, cuff 182, which is annular and hollow, defines and extends between a bottom edge 186 and an opposing top edge 188. Neck 184 extends from top edge 188 of cuff 182 to a top edge 190 of neck 184. An open cavity 192 extends through adapter 180, more specifically, from top edge 190 to bottom edge 186. Neck 184 defines an outside surface 198 with threads 200 sized and shaped to rotatably couple with coupling sleeve 124 of breastpump apparatus 20, for example, with coupling sleeve 124, which would, in other systems, receive a bottle or other hard-sided storage container (not shown).

Cuff 182 has a larger outer diameter than neck 184 and defines an outside surface 196. Adapter 180 includes two or more coupling members 202, which are substantially identical to coupling member 14, as described above, but may include a more permanent or other fastening agent than adhesive 98 (FIG. 8) of coupling member 14. Coupling member 202 is adhered to or otherwise coupled to outside surface 196 of adapter 180, rather than directly to breastpump apparatus 20 as described with respect to coupling member 14. In one example, at least two coupling members 202 are used, with one coupling member 202 being secured to each of opposing sides of cuff 182 to facilitate coupling of both flaps 60 and 62 of storage bag 12 to cuff 182. In this manner, when a consumer wishes to pump directly into storage bag 12, adapter 180 is used and coupling components 16 of storage bags 12 are selectively secured to coupling members 202. When a consumer wishes to pump into a bottle or other container, adapter 180 is easily removed with no residual of coupling member 202 remaining on breastpump apparatus 20. While coupling members 202 are shown of an example length in FIGS. 14 and 15, in other examples, one or more coupling member 202 may extend around part or an entirety of a perimeter of cuff 182, as generally illustrated with broken lines 208. Where only one coupling member 202 is used, coupling member 202 extends at least about 180 degrees around an outer perimeter of cuff 182.

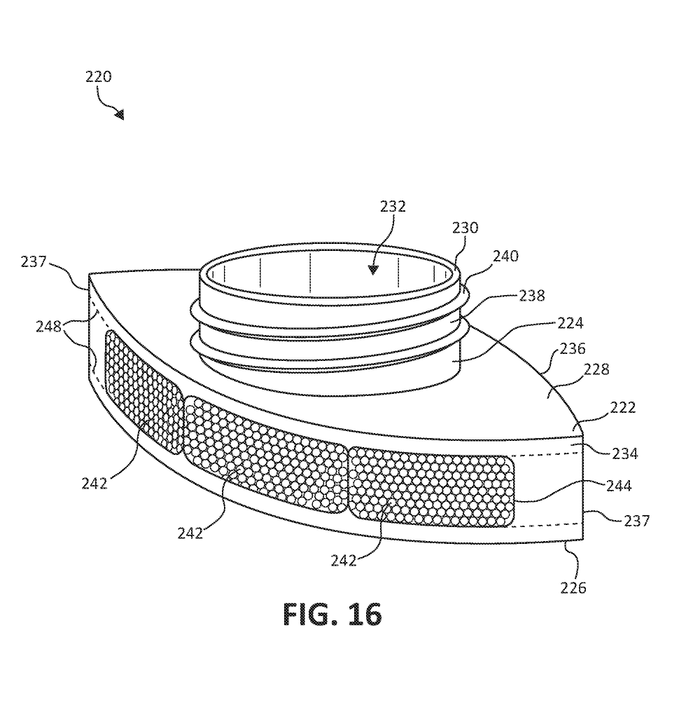

FIG. 16 illustrates one embodiment of an adapter 220, which is an alternative to adapter 180, includes a cuff 222 and a neck 224 coaxially positioned and extending upwardly from cuff 222. More specifically, in one example, cuff 222 is shaped as a vesica piscis with arced side surfaces 234 and 236 converging at vertical edges 237. Each of arced side surfaces 234 and 236 has a width, measured from one vertical edge 237 to the other, that is substantially equal to a width of one of front wall 22 and rear wall 24 (see FIG. 1) or at least a width as measured along interior surfaces 28 or 32, respectively, between side seams 36. Cuff 222 is hollow and extends between a bottom edge 226 and an opposing top edge or shoulder 228 extending inwardly from tops of arced side surfaces 234 and 236.

Neck 224 has a smaller width and length than cuff 222 and extends from shoulder 228 of cuff 222 to a top edge 230 of neck 224. An open cavity 232 extends through adapter 220, more specifically, from top edge 230 of neck 224 to bottom edge 226 of cuff 222. Neck 224 defines an outside surface 238 with threads 240 sized and shaped to rotatably couple with coupling sleeve 124 of breastpump apparatus 20, for example, with coupling sleeve 124, which would, in other systems, receive a bottle or other hard-sided storage container (not shown).

Adapter 220 includes two or more coupling members 242, which are substantially identical to coupling member 14, as described above, but may include a more permanent adhesive (not shown) or other fastening agent than adhesive 98 (FIG. 8) of coupling member 14. Coupling members 242 are each secured to one of arced side surfaces 234 and 236 of adapter 180, rather than directly to breastpump apparatus 20 as described with respect to coupling member 14. In one example, at least two coupling members 242 are used with at least one coupling member 242 being secured to each of opposing arced side surfaces of cuff 182 to facilitate coupling of both flaps 60 and 62 of storage bag 12 (FIGS. 1-4) to cuff 222. In one example, multiple coupling members 242 and/or longer coupling members 242 than illustrated are applied to each of arced side surfaces 234 and 236.

In one example, one or more coupling members 242 extend around a substantially entirety or at least about 180 degrees of an outer perimeter of cuff 222 as generally indicated with dashed lines 248. In this manner, when a consumer wishes to pump directly into storage bag 12, adapter 220 is used, in a similar manner as described and illustrated for adapter 180, and coupling components 16 of storage bags 12 are selectively secured to coupling members 242. When a consumer wishes to pump into a bottle or other container, adapter 220 is easily removed with no residual of coupling member 202 remaining on breastpump apparatus 20.

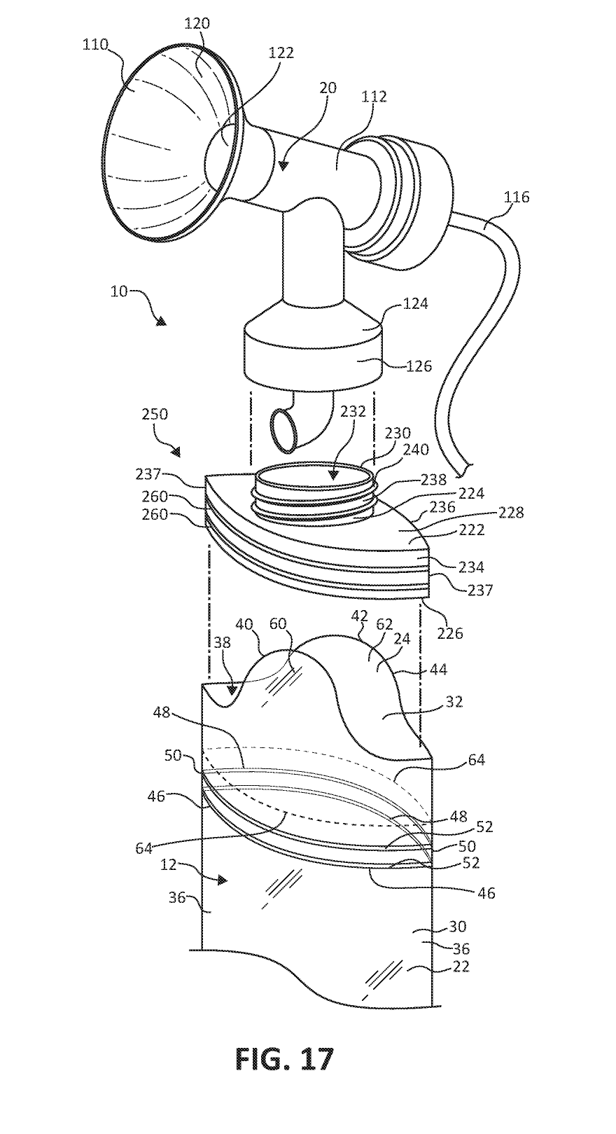

FIG. 17 illustrates breastpump apparatus 20 and storage bag 12 being used with one embodiment of an adapter 270. Adapter 270 is formed substantially identically to adapter 220 except the at least one coupling member 242 is replaced with closure strip elements 260. Each closure strip element 260 is similar to one of closure strip elements 46 and 48 of storage bag 12. More specifically, in one example, closure strip elements 260 on first arced side surface 234 are substantially identical to closure strip elements 48 of rear wall 24 of storage bag 12, and closure strip elements 260 on second arced side surface 236 are substantially similar to closure strip elements 46 of front wall 22 of storage bag 12, where closure strip elements 46 and 48 are complimentary and couple with one another to securely close storage bag 12 in a liquid tight manner.

During use, storage bag 12 is aligned with adapter 250 and closure strip elements 46 of front wall 22 are zip sealed to closure strip elements 260 on first arced side surface 234, and closure strip member 48 of rear wall 24 are zip sealed to closure strip elements 260 on second arced side surface 236. In this embodiment, storage bag 12 is selectively sealed to adapter 250 around a substantial entirety of top opening 44 providing an even more spill proof environment for expression of breast milk 18. In one example, coupling components 16 are eliminated from storage bag 12 when storage bag 12 is configured for use with adapter 250. Once filled, storage bag 12 is removed from adapter 250 and closure strip elements 46 and 48 are secured to each other to close storage bag 12 in a liquid tight manner as will be apparent to those of skill in the art reading the present application.

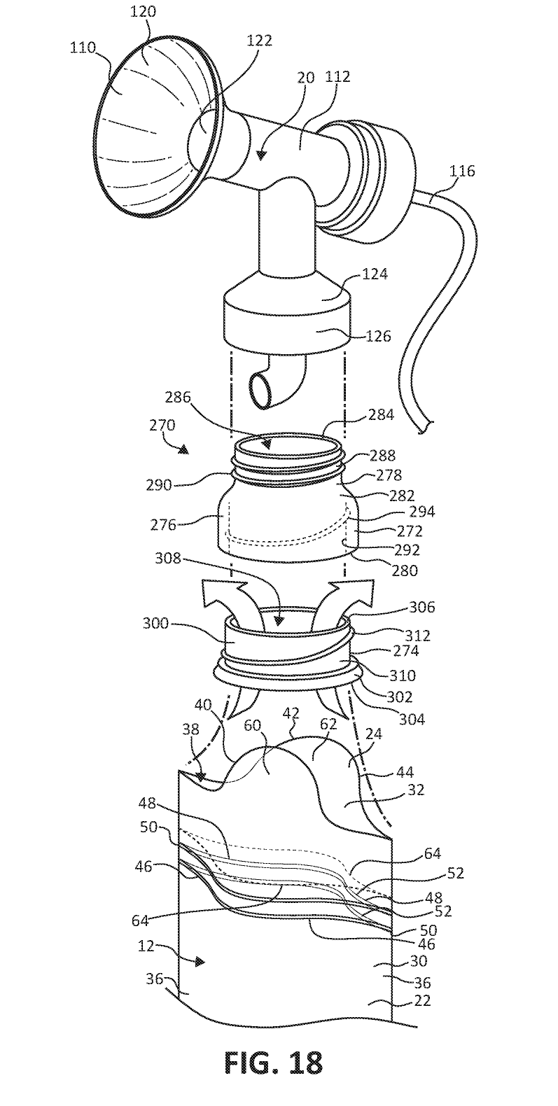

Yet another adapter 270 is illustrated for use with breastpump apparatus 20 and storage bag 12 in FIG. 18. Adapter 270 is a two-piece system including an adapter sleeve 272 and an adapter base 274 configured to receive flaps 60 and 62 of storage bag 12 between them before adapter sleeve 272 is secured to breastpump apparatus 20. Sleeve 272, in one example, includes a cuff 276 and neck 278, which is coaxially positioned relative to and extends upwardly from cuff 276. More specifically, in one example, cuff 276, which is annular and hollow, defines and extends between a bottom edge 280 and an opposing top edge or shoulder 282. Shoulder 282 narrows to neck 278, which extends from shoulder 282 of cuff 276 to a top edge 284 of neck 278. An open cavity 286 extends through adapter 270, more specifically, from top edge 284 to bottom edge 280. Neck 278 defines an outside surface 288 with threads 290 sized and shaped to rotatably couple with breastpump apparatus 20, for example, with coupling sleeve 124, which would, in other systems, receive a bottle or other hard-sided storage container. Adapter sleeve 272 further defines at least one internal thread 294 extending around a portion of open cavity 286 corresponding with cuff 276.

Adapter base 274 includes a cylinder 300 and an annular rim 302. Cylinder 300 defines a top edge 306 and extends downwardly from top edge 306 to annular rim 302. Cylinder 300 additionally defines an outside surface 210 with threads 312 extending therearound. Cylinder 300 and threads 312 are sized and shaped to rotably mate with adapter sleeve 272 via thread 294. Annular rim 302 extends radially outward and downwardly from outside surface 210 and, in one embodiment, has a greater outside diameter than an outside diameter of cuff 276. Adapter base 274 includes an open channel 308 extending therethrough, from top edge 306 to bottom edge 204.

During use, flaps 60 and 62 are pulled through open channel 308 from below annular rim 302 to extend out of and fold around cylinder 300 as generally indicated by the enlarged arrows in FIG. 18. More specifically, flaps 60 and 62 fold over to cover threads 312. Adapter sleeve 272 is placed over adapter base 274 and is rotated relative to adapter base 274 mating thread 312 of adapter base with thread 294 of adapter sleeve 272 even with flaps 60 and 62 being maintained between thread 312 and thread 294. Adapter sleeve 272 is rotatably secured to cuff 276 of breastpump apparatus 20 for use and expression of breast milk 18 (see e.g., FIG. 3) from breastpump apparatus 20 into storage bag 12 through adapter 270. Other adapters using concepts similar to those described above and permitting expression of milk from breastpump apparatus 20 into storage bag 12, such as a bag, are al so contemplated.

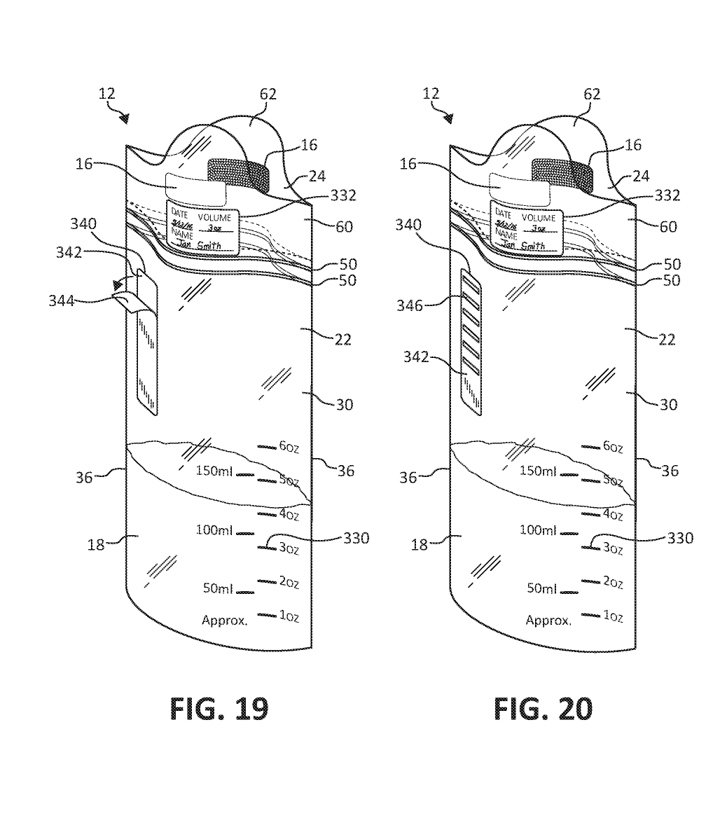

FIGS. 19 and 20 illustrate storage bag 12 with additional features according to embodiments of the present invention. Any one or more of the additional features may be incorporated into each storage bag, or none of the additional features may be included on given storage bag 12. In one example, front wall 22 includes measurement indicia 330 for facilitating a consumer in approximating how much breast milk 18 is maintained within storage bag 12. As shown in FIGS. 19 and 20, the opaque breast milk 18 as viewed through the substantially transparent front wall 22 provides a top liquid line viewed relative to measurement indicia 330 to indicate a fill level for storage bag 12.

Storage bag 12 may also include a writable field, such as an opaque field 332 providing spaces for consumer to write information about breast milk 18 stored therein. For example, opaque field 332 may provide spaces for consumer to record a date breast milk 18 was collected, a volume of breast milk 18 in the corresponding storage bag 12, and a name of the baby or mother associated with breast milk 18 for identification and proper use of breast milk 18. Other types of information may also be recorded to opaque field 332 as will be understood by those of skill in the art upon reading this application.

In one embodiment, storage bag 12 includes sensor field 340 for sensing time, temperature, freshness, or other variable of importance to maintaining breast milk 18 in a safe manner. As illustrated, sensor field 340 is elongated and is substantially planar, applied to exterior surface 30 of front wall 22 in a secure manner. Sensor field 340 may be provided to consumer previously coupled to storage bag 12. In one embodiment, sensor field 340 is provide to consumer as a separate sticker or label that consumer applies to storage bag 12 for use. In one example, where sensor field 240 measures time, sensor field 340 is initially covered by peel strip 344, which keeps sensor field 340 in a manner not directly exposed to an external environment.

When a consumer first places either a fresh supply of breast milk 18 or a frozen supply of breast milk on a counter or in a refrigerator, he or she peels away peel strip 344 exposing sensor field 340 therebelow to the surrounding environment, which activates sensor field 340. Sensor field 340 is configured to change a visual display thereon as time passes. For instance, where breast milk 18 is determined to remain fresh in an unrefrigerated state for at least six hours, sensor field is configured to display a new bar or other suitable demarcation 346 as predetermined periods of time pass, that is, as sensor field 340 remains exposed to a surrounding environment, indicating to a consumer how long the breast milk 18 has been maintained in an unrefrigerated manner. For example, a new demarcation 346 appears in sensor field 340 every half hour or every hour that sensor field 340 is exposed to the surrounding environment. In one embodiment, the colors of each additional demarcation 346 increases in intensity as the demarcation nears an indication that breast milk 18 in storage bag 12 has been exposed to its surroundings for an undesirably long length of time. This indication of time gives a consumer peace of mind, helping consumer to keep track of how long breast milk has been kept in certain conditions. In another embodiment, demarcations 346 may appear every 12 hours that sensor field 340 is exposed to refrigerated embodiment, allowing a consumer to track refrigerated time for breast milk 18. In yet another embodiment, sensor field 340 may include a temperature component such that demarcations 346 appear faster at room temperature than at cooler, e.g., refrigerated, temperatures for even broader use. Other periods of time inducing a new demarcation 346 are also contemplated.

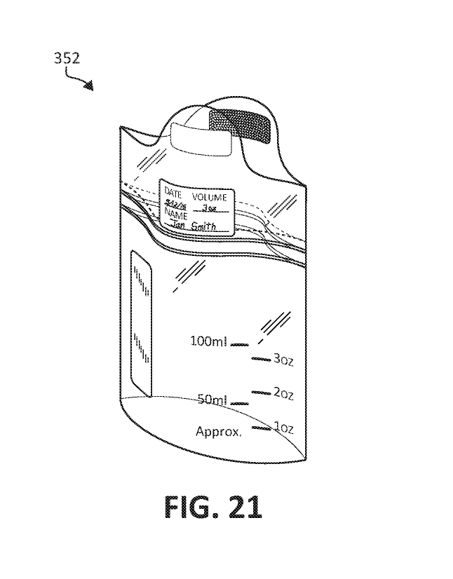

Other variations of storage bag 12 are also contemplated. For example, storage bags 12 may be available in a number of storage capacities, such as the six-ounce capacity of storage bag 12 and/or the three-ounce capacity of storage bag 352, illustrated in FIG. 21, which other than storage capacity an size, is substantially identical to storage bag 12.

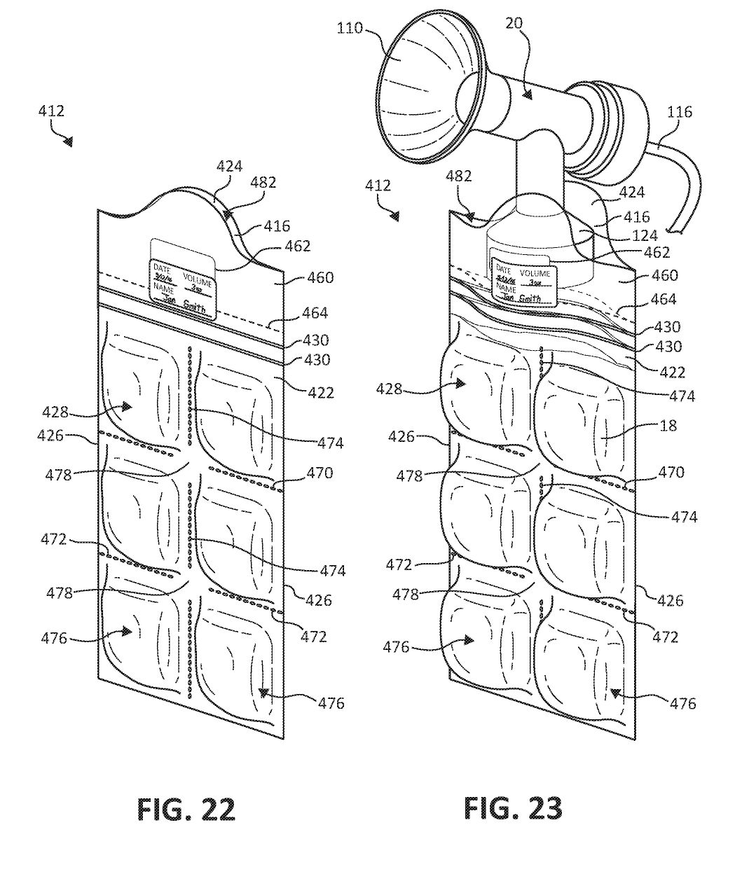

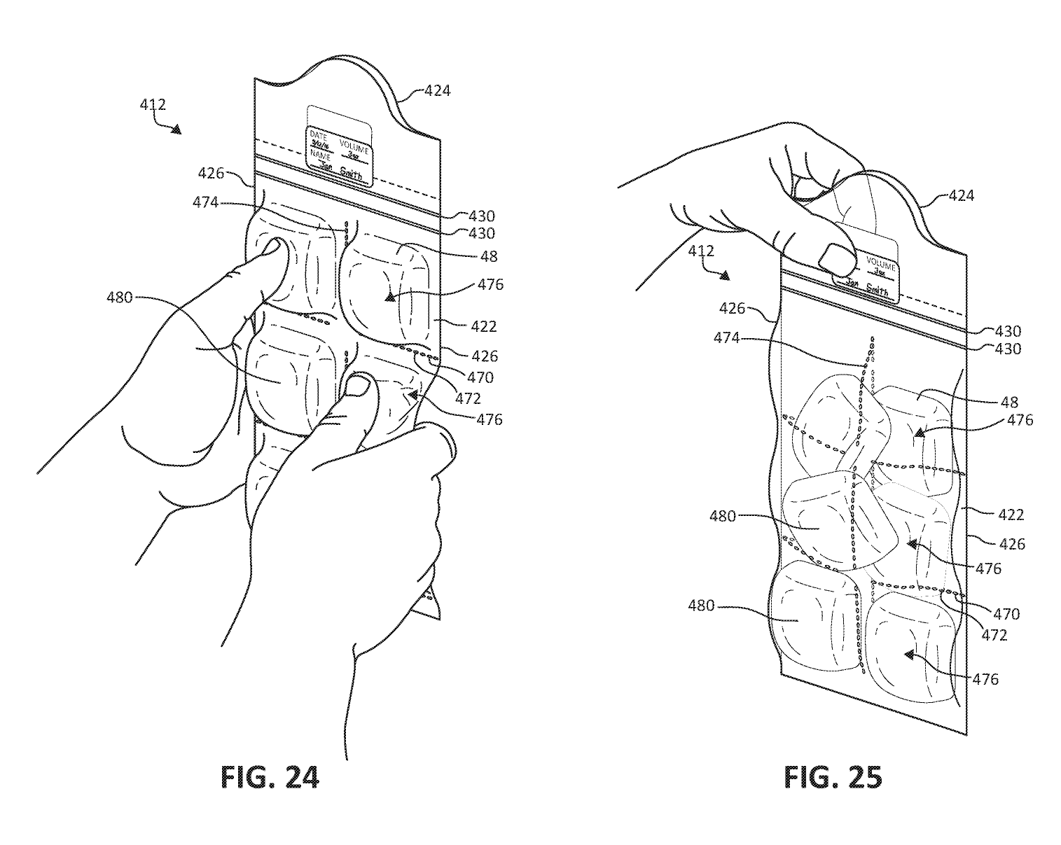

FIG. 22 illustrates yet another storage bag 412 for use with breast milk collection system 10 (see FIG. 1). Storage bag 412 includes a coupling component 416, a front wall 422, a rear wall 424, opposing side seams 426, closure strips 430, flaps 460 and 462, perforation line 464, and a top opening 482 that are substantially identical to coupling component 16, front wall 22, rear wall 424, side seams 36, closure strips 50, flaps 60 and 62, perforation line 64, and top opening 44 of storage bag 12 (see, e.g., FIG. 1), except where specifically described otherwise below. Storage bag 412 defines a storage chamber 428 similar to storage chamber 38; however, storage chamber 428 of storage bag 412 is divided into smaller individual chambers 476 each having a substantially equal capacity for forming a number of substantially identically sized, small breast milk cubes 480 within a single storage bag 412. In one example, each individual chamber 476 has a storage capacity of one ounce or more. In one embodiment, storage bag 412 may form individual chambers 476 of different capacities, such as a one individual chamber 476 having a one ounce capacity while another individual chamber has a capacity of two ounces.

For example, storage apparatus 412 includes releasable interior seals 470, including one or both of horizontal seals 472 and vertical seals 474, in one embodiment. Each interior seal 470 is formed between front wall 422 and rear wall 424 holding front wall 422 and rear wall 424 together in a liquid tight manner at interior seal 470. In FIG. 22, interior seals 470 are generally arrange in a grid like manner, with vertical seals 474 each being substantially linear, linearly aligned with the other vertical seals 474, and being separated from adjacent ones of vertical seals 474 by a seal gap 478. Horizontal seals 472 are substantially linear and extend in linearly aligned pairs that are vertically spaced from other pairs of horizontal seals 472. Each horizontal seal 472 is spaced from the other horizontal seal 472 in the pair by a seal gap 478. Other arrangements of interior seals 470 and seal gaps 478 are contemplated, so long as the location and size of seal gaps 478 allows breast milk 18 to flow from a top opening of storage bag 412 down into each individual chamber 476 of storage bag 412 via gravity, filling each individual chamber 476.

For example, as shown in FIG. 23, in one example, breastpump apparatus 20 is secured to storage bag 412 via coupling component 416, and breastpump apparatus 20 releases breast milk 18 into storage chamber 428. Due to gravity, breast milk 18 travels through seal gaps 478 moving downwardly from breastpump apparatus 20 filing bottom ones of individual chambers 476 first and top ones of individual chambers 476 last. In one example, at least some of individual chambers 476 is filed to its liquid capacity with breast milk 18 during use.

Once storage bag 412 is filled to a desired level, preferably, but not necessarily, filled until each individual chamber 476 is substantially filled, breastpump apparatus 20 is uncoupled from and moved away from storage bag 412, and storage bag 412 is sealed via closure strips 430. In one embodiment, even when individual chambers 476 are filled with breast milk 18, storage bag 412 remains substantially flat as opposed to bulky in depth. Filled storage bag 412 is frozen in either a horizontal manner or in a vertical position illustrated in FIGS. 22 and 23 to create a plurality of breast milk cubes 480, one in each individual chamber 476. When consumer wishes to use one of breast milk cubes 480, consumer grasps storage bag 412, as generally shown in FIG. 25, and manipulates the same by pushing side seams 36 toward one another and flaps 460 and 462 and bottom of storage bag 412 with sufficient force to cause interior seals 470 to release. As interior seals 470 release, front wall 422 is released from interior portions of rear wall 424, in turn, opening storage chamber 428 as a single chamber with no individual chambers 476 therein, as illustrated in FIG. 25. The resulting open storage chamber 428 is filled with individual breast milk cubes 480 loosely maintained therein. In this state, the consumer is easily able to open storage bag 412, and individually remove the one or more breast milk cubes 480 for use. The smaller breast milk cubes 480 are advantageous in a number of situations, including for use with the milk of another storage bag 12 that either was not fully filled and/or does not include enough breast milk for a typical single feeding of a given infant, for use where an infant has consumed a typical amount for a single feeding, but remains hungry, among others.

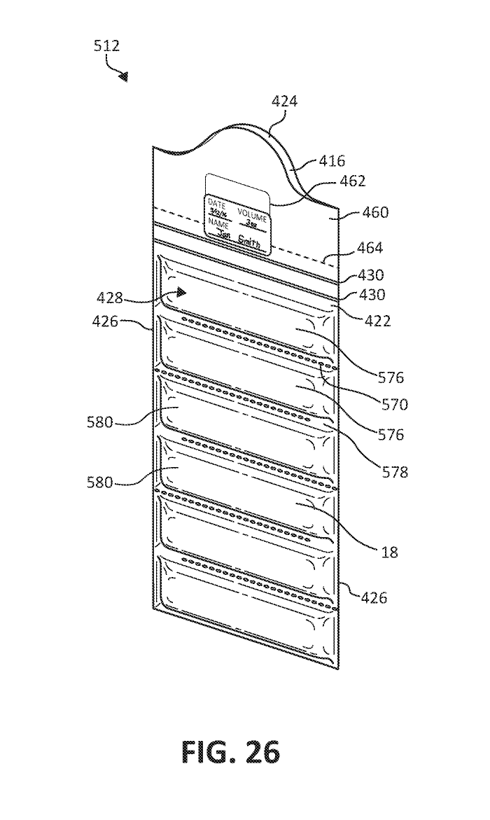

FIG. 26 illustrates another storage bag 512 for forming smaller breast milk cubes 580, that is, substantially identical to storage bag 412 other than features defining the breast milk cubes 580. Storage bag includes interior seals 570 that are each substantially linear, are each horizontal, and/or each run substantially parallel to each other. Each interior seal 570 is formed along a substantially entirety of a width of storage bag 512, but for seal gaps 578 ends of interior seals 570 along alternating ones of side seams 426, to form elongated horizontally extending individual chambers 576. Breast milk 18 fills the bottommost individual chamber 576 first due to gravity. Filled storage bag 512 is used substantially identically to storage bag 412 to produce a plurality of breast milk cubes 580 in storage bag 512. The elongated and narrow nature of breast milk cubes 580 is beneficial, as it allows individual ones of breast milk cubes 580 to be easily placed into a bottom through the bottle neck thereof, even if the bottle neck is quite narrow, prior to thawing. Other shapes of breast milk cubes and/or configurations of interior seals are also contemplated.

Although the invention has been described with respect to particular embodiments, such embodiments are meant for illustrative purposes only and should not be considered to limit the invention. Various alternatives and changes will be apparent to those of ordinary skill in the art upon reading this application. Other modifications within the scope of the invention and its various embodiments will be apparent to those of ordinary skill.

What is claimed is:

1. A breast milk collection system comprising:
a storage bag including:
a storage chamber,
a closure strip for selectively closing a top of the storage chamber,
two bag flaps each extending from the closure strip away from the storage chamber, and
two coupling components each coupled to a different one of the two bag flaps, and each of the two coupling components includes a plurality of flap protuberances; and
at least one coupling member formed separately from the storage bag and defining a first side and a second side, the first side including a plurality protuberances extending therefrom, and the second side being configured to be coupled to a breastpump apparatus;
wherein at least one of the two coupling components selectively couples with the at least one coupling member via interaction between the plurality of flap protuberances of the at least one of the two coupling components and the plurality of protuberances of the at least one coupling member when of the at least one of the two coupling components and the at least one coupling member are pushed together.

2. The breast milk collection system of claim 1, wherein the plurality of protuberances of the at least one coupling member are hook shaped.

3. The breast milk collection system of claim 2, wherein the plurality of protuberances of the at least one coupling member are each shaped substantially identical to the plurality of flap protuberances of the two coupling components as hooks such that the at least one coupling member and the at least one of the two coupling components collectively form a hook-and-hook fastener.

4. The breast milk collection system of claim 1, wherein each of the plurality of flap protuberances includes a shaft extending from one of the two bag flaps and a rail extending outwardly from the shaft opposite the one of the two bag flaps.

5. The breast milk collection system of claim 1, wherein the plurality of flap protuberances of each of the two coupling components are integrally formed with the different ones of the two bag flaps.

6. The breast milk collection system of claim 1, wherein the at least coupling member includes a first coupling member and a second coupling member, the first coupling member being coupled with one of the two coupling components, and the second coupling member being coupled with the other one of the two coupling components.

7. The breast milk collection system of claim 6, in combination with the breastpump apparatus, wherein:
the breastpump apparatus includes a breast shield and a coupling sleeve,
the coupling sleeve is configured to threadably couple with a bottle,
the first coupling member is secured to one side of the coupling sleeve of the breastpump apparatus, and
the second coupling member is secured to the other side of the coupling sleeve such that the storage bag hangs from the coupling sleeve in an open manner via the first coupling member and the second coupling member such that breast milk from the breastpump apparatus will be expressed into the storage bag.

8. The breast milk collection system of claim 1, wherein the plurality of flap protuberances of each of the two bag flaps extends from one of the two bag flaps toward the other of the two bag flaps.

9. The breast milk collection system of claim 1, wherein the second side of the at least one coupling member is at least partially covered with adhesive for selective application of the at least one coupling member to the breastpump apparatus.

10. The breast milk collection system of claim 1, further comprising:
an adapter configured to be selectively coupled with the breastpump apparatus, the adapter including a cuff and a neck, the neck is threaded and sized to threadably couple with the breastpump apparatus, the cuff extends outwardly from a bottom of the neck defining an outside surface, and the at least one coupling member is secured to the outside surface of the cuff.

11. The breast milk collection system of claim 10, in combination with the breastpump apparatus, wherein:
the breastpump apparatus includes a breast shield and a coupling sleeve,
the neck is received within and threadably coupled with the coupling sleeve, and
the at least one of the two coupling components selectively coupled with the at least one coupling member such that the storage bag is suspended from the adapter.

13. The breast milk collection system of claim 10, wherein the cuff of the adapter is substantially cylindrical.

14. The breast milk collection system of claim 10, wherein the cuff of the adapter is substantially shaped as a vesica piscis.

15. The breast milk collection system of claim 1, wherein the coupling component extends across less than half of an outside width of the storage bag measured between two opposing sides of the storage bag.

16. The breast milk collection system of claim 1, wherein the storage bag includes a perforation line on each flap, the perforation line extending substantially parallel to the closer strip between the closure strip and the one of the two coupling components positioned on the same flap as the perforation line.

17. The breast milk collection system of claim 1, wherein the storage bag includes a sensor applied to an external surface of the storage bag.

18. The breast milk collection system of claim 17, wherein the sensor visually indicates passage of time.

19. The breast milk collection system of claim 1, wherein the storage bag includes a plurality of interior seals dividing the storage chamber into a plurality of individual chambers, and each of the plurality of individual chambers are in fluid communication with the others of the plurality of individual chambers.

20. The breast milk collection system of claim 19, wherein the interior seals are configured to be broken without damaging the storage bag when the storage bag is repeatedly squeezed.

21. The breast milk collection system of claim 19, wherein each individual chamber has a one-ounce volumetric capacity.

22. The breast milk collection system of claim 1, further comprising:
a container including a bottom wall, a front wall, an open top opposite the bottom wall, and a door in the front wall adjacent the bottom wall, wherein:
during non-use, the storage bag is one of a plurality of storage bags maintained within the container, and
the storage bags are removable from the container on a first in-first out basis via the door.

23. A breast milk storage bag including:
a storage chamber,
a closure strip for selectively closing a top of the storage chamber,
two bag flaps each extending from the closure strip away from the storage chamber, and
two coupling components each coupled to a different one of the two bag flaps, and each of the two coupling components includes a plurality of flap protuberances extending from one of the bag flaps toward the other one of the two bag flaps as part of a hook-and-hook fastener.
